(19) United States
(12) Patent Application Publication
(10) Pub. No.: US 2005/0176031 A1
(43) Pub. Date: Aug. 11, 2005

(54) Kinship analysis program for missing persons and mass disaster

(76) Inventors: Christopher P. Sears, Watertown, MA (US); Viviane Siino, Watertown, MA (US)

Correspondence Address: Viviane Siino, 25 Paul St, Watertown, MA, 02472, US

(21) Appl. No.: 10/974,174

(22) Filed: Oct. 27, 2004

Related U.S. Application Data
(60) Provisional application No. 60/525,223, filed on Oct. 30, 2003.

(51) Int. Cl. C12Q 1/68; G06F 19/00; G01N 33/48; G01N 33/50

(52) U.S. Cl. 435/6; 702/20

(57) ABSTRACT

A kinship analysis program for missing persons and mass disasters for kinship and missing persons analyses. The inventive device includes consensusG, Kinship Indices, Matching, cross-checking, pedigree that performs the following: Extract a list of unique victims' genotypes from the remains or missing persons database, make perfect matches to personal effect, identify next-of-kin related to a victim, make matches through parentage trios, confirm consistency of family pedigrees, exclude nearly all fortuitous hits on the basis of the SM scoring performance of the entire family of the relative that triggered the fortuitous hit, make matches despite the known existence of errors in sample names and reported relationships, flag samples with inconsistent reported relationships, display matches in their context, so that “close-calls” are brought to the attention of the data reviewer. An algorithm which reduces very large collections of complete/partial STR (Short Tandem Repeats) genotypes derived from the remains down to a restricted number of consensus genotypes believed to reflect the many different victims. An algorithm which calculates Kinship Indices through a segregated score approach and likelihood ratio calculations through a linkage to the KinTest software. An algorithm which contrasts the consensus genotypes against the data set of genotypes from next-of-kin and personal effects for direct matches to personal effects, for evidence of genetic relatedness to kin through the Kinship Indices, or for successful production of a parentage trio with any two members of the next-of-kin cohort. An algorithm which cross-checks matches to parentage trios against other kinship scoring results between the victim and other members of the same family to confirm that the purported family structure is consistent with Mendelian inheritance rules. A schematic diagram describing the relationship of the relatives to a matched victim. A user interface that is accessible to a broader range of users.

### CROSS REFERENCE TO RELATED APPLICATION

Priority is herewith claimed under 35 U.S.C. § 119(e) from Provisional Patent Application 60/525,223, filed Oct. 30, 2003 which is incorporated by reference in its entirety.

### FIELD OF THE INVENTION

The present invention relates generally to a kinship analysis application and more specifically it relates to the identification process for missing persons and mass disasters.

### BACKGROUND OF THE INVENTION

DNA information can be isolated and used as a means of identifying living organisms including human beings. Unlike a conventional fingerprint which can be modified with surgery, an individual's DNA is the same for every cell, tissue, and organ. It cannot be altered by any known treatment. Consequently, DNA is rapidly becoming the primary method for identifying and distinguishing among individuals.

It can be appreciated that kinship analyses have been in use for years. Typically, kinship analysis programs are comprised of a component which obtain trios by a two-step algorithm that tests all possible parentage trio combinations for any given victim of a Mass Disaster. Intelligent agents that perform these analyses have not been available until now.

The main deficiencies with conventional kinship analyses as they currently stand are:

1. Limited usability. Existing methods are limited to a small number of experts in the field as they require extensive experience with forensic, statistical and genetics principles.

2. Inconvenient and inflexible. Even to experts in the field existing methods are limited to use in particular situations and cannot be generalized to and disaster or incident.

3. Limited growth potential. Current methods are not equipped to sustain datasets of the size required for missing persons identification.

4. Limited exposure. Another problem with conventional kinship analysis applications is they are not commercially deployable solutions.

5. Absence of pedigree visualization. Existing methods do not include pedigree generation tools that facilitate visualization of a prospective victim's place within a selected family in a manner that is intuitive to analysts.

6. Lacking connectivity. Existing methods are unable to make use of networking between computers to facilitate the tasks of data collection, analysis, and identification.

While these applications may be suitable for the particular purpose to which they address, they are not as suitable for kinship and missing persons analyses involving large volumes of data.

In these respects, the kinship analysis program for missing persons and mass disasters according to the present invention substantially departs from the conventional concepts and designs of the prior art, and in so doing provides an apparatus primarily developed for the purpose of kinship and missing persons analyses.

### SUMMARY OF THE INVENTION

In view of the foregoing disadvantages inherent in the known types of Kinship analysis methods now present in the prior art, the present invention provides a new kinship analysis software program for missing persons and mass disasters construction wherein the same can be utilized for kinship and missing persons analyses.

The general purpose of the present invention, which will be described subsequently in greater detail, is to provide a new kinship analysis program for missing persons and mass disasters that has many of the advantages of the kinship analysis methods mentioned heretofore and many novel features that result in a new kinship analysis program for missing persons and mass disasters which is not anticipated, rendered obvious, suggested, or even implied by any of the prior art kinship analysis applications, either alone or in any combination thereof.

To attain this, the present invention generally comprises consensus views, Kinship Indices, matching, cross-checking and pedigree components that perform the following: extract a list of unique victims' genotypes from the remains or missing persons dataset, make perfect matches to personal effect, identify next-of-kin related to a victim, make matches through parentage trios, confirm consistency of family pedigrees, exclude nearly all fortuitous hits on the basis of the SM scoring performance of the entire family of the relative that triggered the fortuitous hit, make matches despite the known existence of errors in sample names and reported relationships, flag samples with inconsistent reported relationships, display matches in their context, so that “close-calls” are brought to the attention of the data reviewer.

The present invention generally comprises genetic data integration, data visualization, data annotation, and data storage/management modules, and provides a unifying interface for genetic data from divergent sources, to perform the following tasks:

1. Manage data sets and administrate user permissions.

2. Combine very large collections of complete/partial STR (Short Tandem Repeats), mtDNA (mitochondrial DNA), and SNP (single nucleotide polymorphism) genotypes derived from the remains into a restricted number of consensus genotypes believed to reflect the many different victims.

3. Calculate Kinship Indices through a segregated score approach and likelihood ratio calculations using algorithms comparing genetic information of remains and purported family members.

4. Compare the consensus genotypes against the data set of genotypes from next-of-kin and personal effects for direct matches to personal effects, for evidence of genetic relatedness to kin through the Kinship Indices, or for successful production of a parentage trio with any two members of the next-of-kin cohort.

5. Cross-check matched parentage trios against other kinship scoring results between the victim and other members of the same family to confirm that the purported family structure is consistent with Mendelian inheritance rules.

6. Dynamically generate a schematic diagram describing the relationship of the relatives to a matched victim remains.

7. Provide a user interface that is accessible to a broader range of users.

8. Manage server and client computing functions so as to protect sample data on incident sites and to facilitate widespread use of data sources.

9. Distribute processing of sample data across multiple computing machines so as to facilitate and expedite processing of large data volumes.

There has thus been outlined, rather broadly, the more important features of the invention in order that the detailed description thereof may be better understood, and in order that the present contribution to the art may be better appreciated. There are additional features of the invention that will be described hereinafter. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a kinship analysis program for missing persons and mass disasters that will overcome the shortcomings of the prior art devices.

An object of the present invention is to provide a kinship analysis program for missing persons and mass disasters for kinship and missing persons analyses.

Another object is to provide a kinship analysis program for missing persons and mass disasters that is suitable for commercial-grade kinship and missing persons analyses in the form of a desktop and client-server application.

Another object is to provide a kinship analysis program for missing persons and mass disasters that merges linked profiles from mass disaster samples, and connects the profiles to known profiles obtained from relatives of the disaster victims.

Another object is to provide a kinship analysis program for missing persons and mass disasters that assigns typed material in mass disaster cases to one or more possible sources within a limited population of missing or deceased individuals.

Another object is to provide a kinship analysis program for missing persons and mass disasters that contains a CODIS-compatible algorithm that links typed biological material from mass disasters and assigns the material to one or more possible origins within the context of the disaster or event.

Another object is to provide a kinship analysis program for missing persons and mass disasters that contains statistical subroutines that will chart pedigrees and perform analyses, such as likelihood ratios or paternity index, in order to assess the significance of associations obtained from DNA typing results.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 illustrates a flow chart of components relationships in accordance with the invention.

FIG. 2 is a flowchart of the dataset relevant processes in accordance with the invention.

FIG. 3 is an illustration of the load panel in accordance with the invention.

FIG. 4 is an illustration of the administration panel in accordance with the invention.

FIG. 5 is an illustration of the genotype screen in accordance with the invention.

FIG. 6 is an illustration of the priority screen, displaying candidates most likely to produce a match in accordance with the invention.

FIG. 7 is an illustration of the analysis screen in accordance with the invention.

FIG. 8 is an illustration of a schematic ideogram of a family pedigree with corresponding genotyping data report in accordance with the invention.

FIG. 9 illustrates the matching process screen in accordance with the invention.

FIGS. 10A and B is an illustration of the data mapping procedure in accordance with the invention.

FIG. 11 is a pseudo-code description of the reduction algorithm in accordance with the invention.

FIG. 12 is a pseudo-code description of the kinship index algorithm in accordance with the invention.

FIG. 13 is a pseudo-code description of comparison algorithm in accordance with the invention.

FIG. 14 is a pseudo-code description of the data consistency algorithm in accordance with the invention.

### DESCRIPTION OF THE PREFERRED EMBODIMENT

The system and method of the present invention provides a graphical user interface that is capable of kinship analysis for missing persons and mass disasters. Turning now descriptively to the drawings, in which similar reference characters denote similar elements throughout the several views.

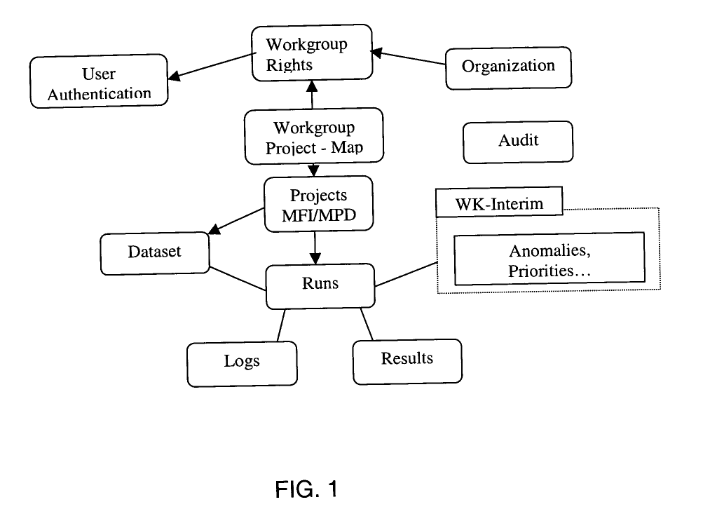

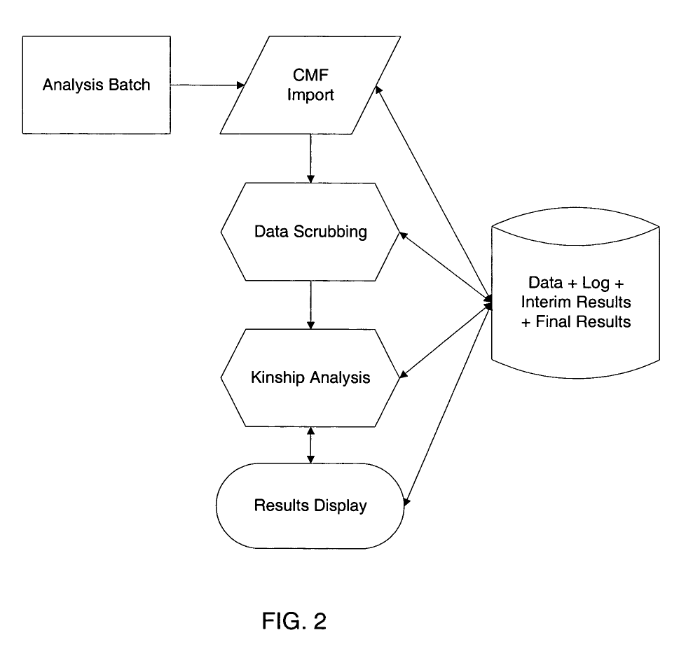

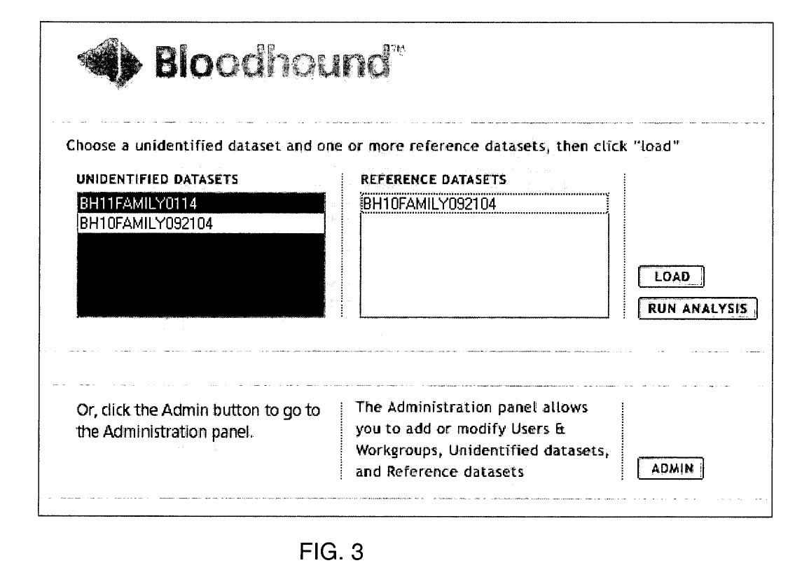

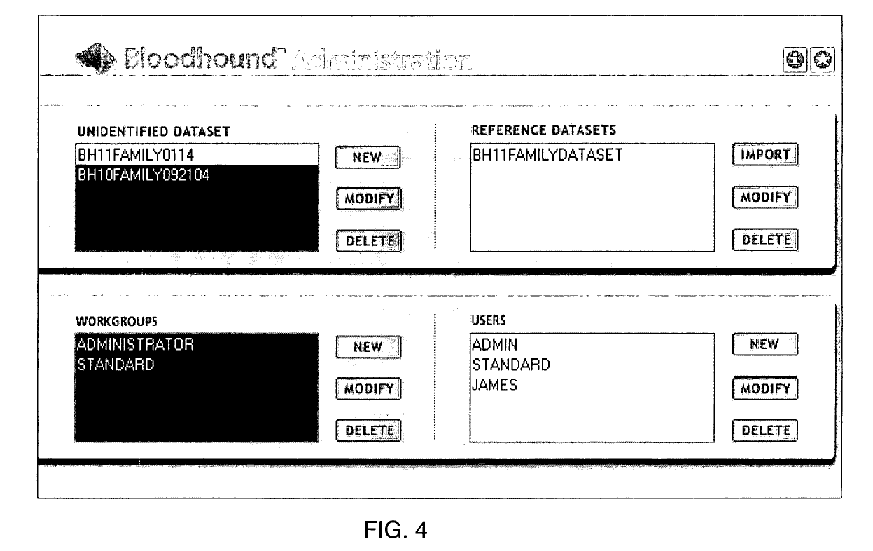

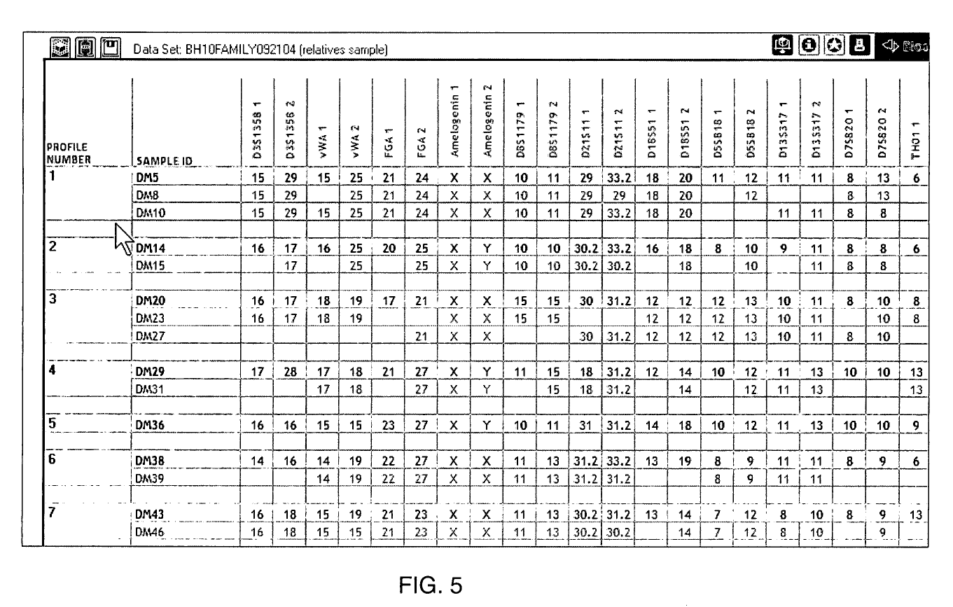

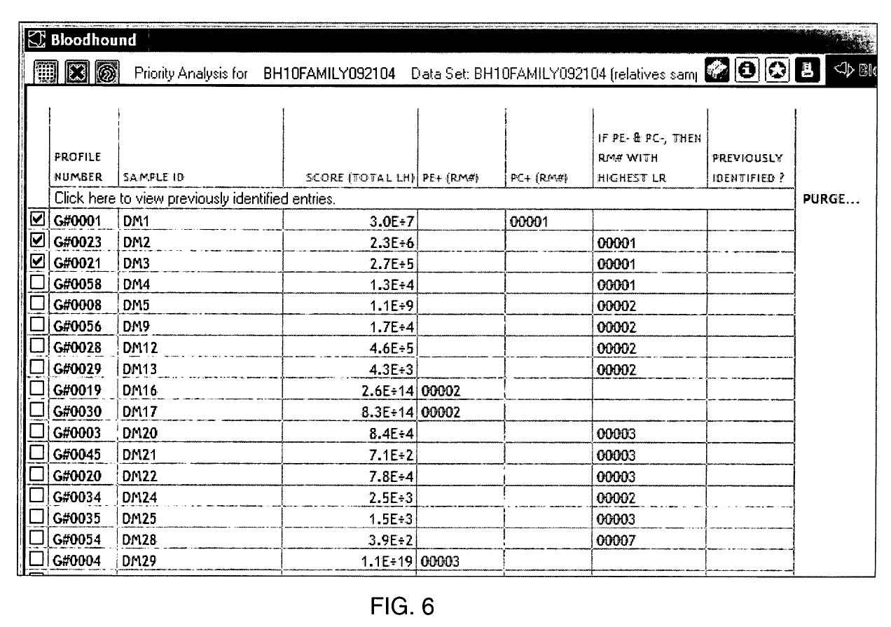

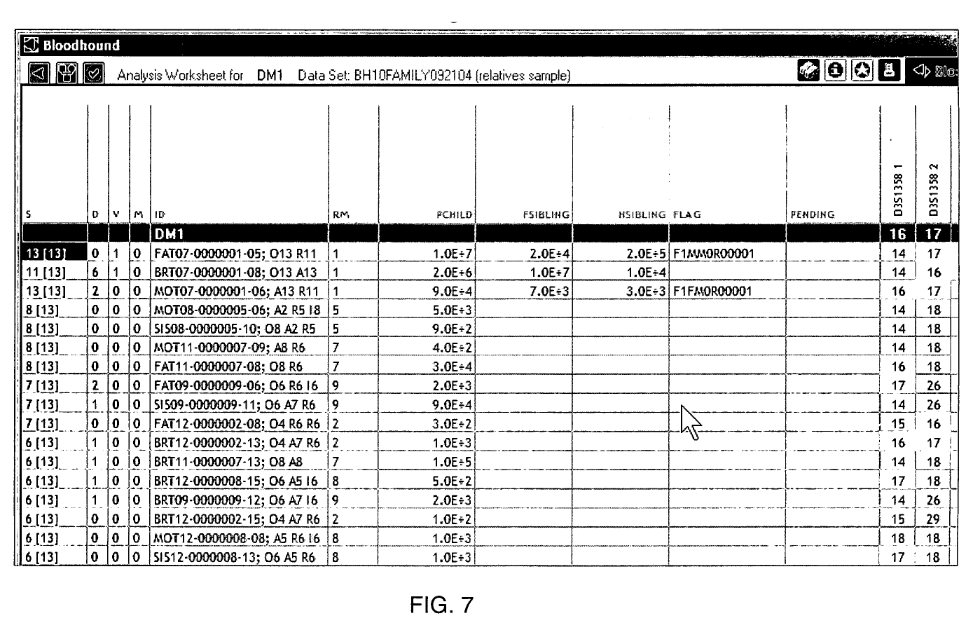

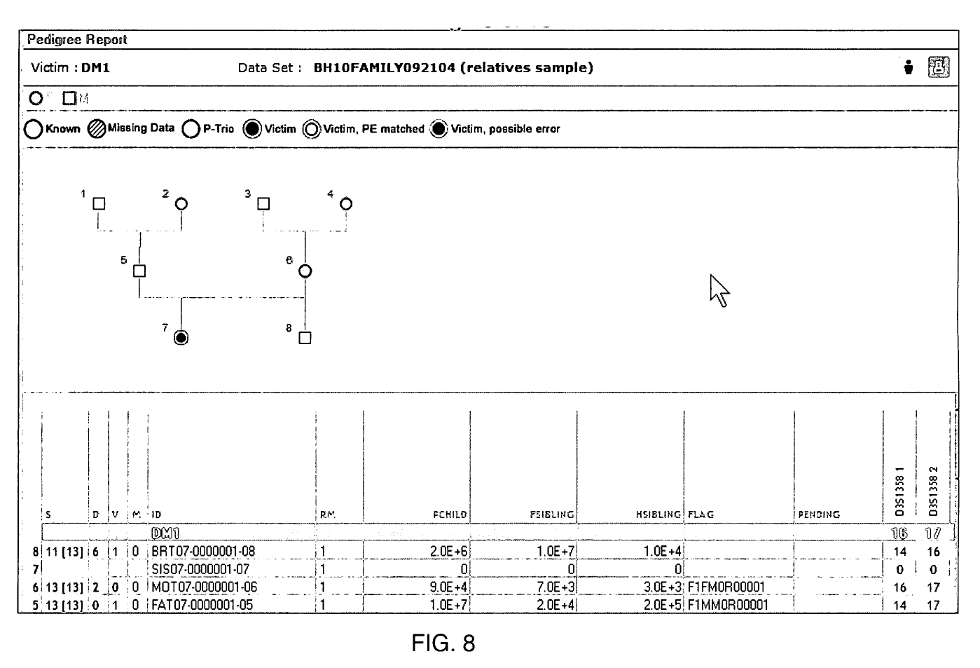

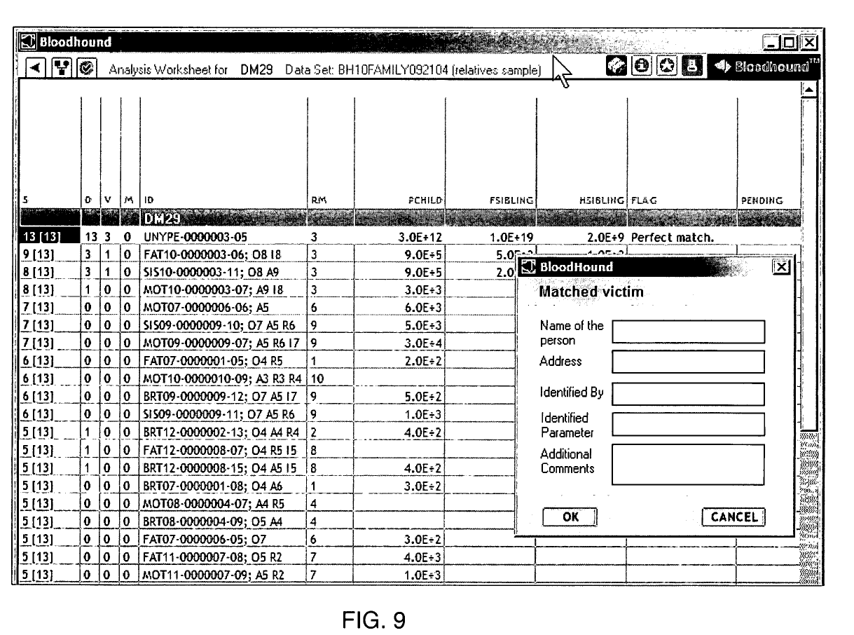

FIG. 1 illustrates a flow chart of components relationships in accordance with the invention. The load panel allows users to view, load, and analyze available datasets based on security levels with an intuitive interface does not require the user to have programming or statistical expertise. FIG. 2 is an illustration of the load panel in accordance with the invention. Each dataset is characterized by the CODIS CMF file selected for import. For a Mass Fatality Incident, the dataset can include victim/relatives. Each dataset can have multiple runs, where a run is defined by an Analysis with its set of logs, results and interim tables. FIG. 3 is a flowchart of the dataset relevant processes in accordance with the invention. The administration panel provides tools to manage sample sets, user permissions, and conduct processing of analyses. FIG. 4 is an illustration of the administration panel in accordance with the invention. The graphical interface provides investigators with organized views of sample data and statistical calculations representing probabilistic relationships to kin. Based on modifiable statistical parameters, the user is able to correlate complete and partial genotype information in order to reduce the number of unique genetic profiles analyzed. FIG. 5 is an illustration of the genotype screen in accordance with the invention. The user can compare the genotypes of remains to those of all reference samples in order to calculate Kinship Indices through a segregated score approach and likelihood ration calculations. The priority screen is intended to quickly allow the user to sort, organize, and focus on the cases that have the highest likelihood of producing identification. FIG. 6 is an illustration of the priority screen, displaying candidates most likely to produce a match in accordance with the invention. The analysis screen provides the genotype details of the selected victim, as well as the best matched reference samples, including the alleles, parentage trio flags, predicted core repeat slip mutations, and relationship scores. FIG. 7 is an illustration of the analysis screen in accordance with the invention. This analysis process is not limited to the nuclear family and can accommodate complex pedigrees. Schematic charts of familial relationships and ancillary data (e.g., familial genotypes and likelihood ratios) are created programmatically on the basis of data input and help to verify the correctness of identifications. FIG. 8 is an illustration of a schematic ideogram of a family pedigree with corresponding genotyping data report in accordance with the invention. When a match is clearly identified the use can mark the sample as such. FIG. 9 illustrates the matching process screen in accordance with the invention.

According to at least one embodiment of the present invention, the application comprises genotype data and statistical algorithms (e.g. STR, mtDNA, SNPs, consensus, Kinship Indices, Matching, cross-checking, pedigree). The algorithms performs the following: Extract a list of unique victims' genotypes from the remains or missing persons dataset, make perfect matches to personal effect, identify next-of-kin related to a victim, make matches through parentage trios, confirm consistency of family pedigrees, exclude nearly all fortuitous hits on the basis of the SM scoring performance of the entire family of the relative that triggered the fortuitous hit, make matches despite the known existence of errors in sample names and reported relationships, flag samples with inconsistent reported relationships, display matches in their context, so that “close calls” are brought to the attention of the data reviewer.

What follows describe elements that compose a graphical interface in accordance with the invention.

1. Data types: The graphical interface provides integrated data on kinship analysis and genetic identification information including, but not limited to STR profiles, mtDNA, SNPs, fingerprinting, dental records and x-ray images.

2. Data integration: Data are provided either by importing into a Relational Database Management System (RDMS), other database, or via TCP/IP or other network link to the user. Databases can be maintained locally or remotely.

3. Data format: The data format is relational database.

4. Data source: Data can be obtained from CODIS Metafiles, or from proprietary data maintained by the user.

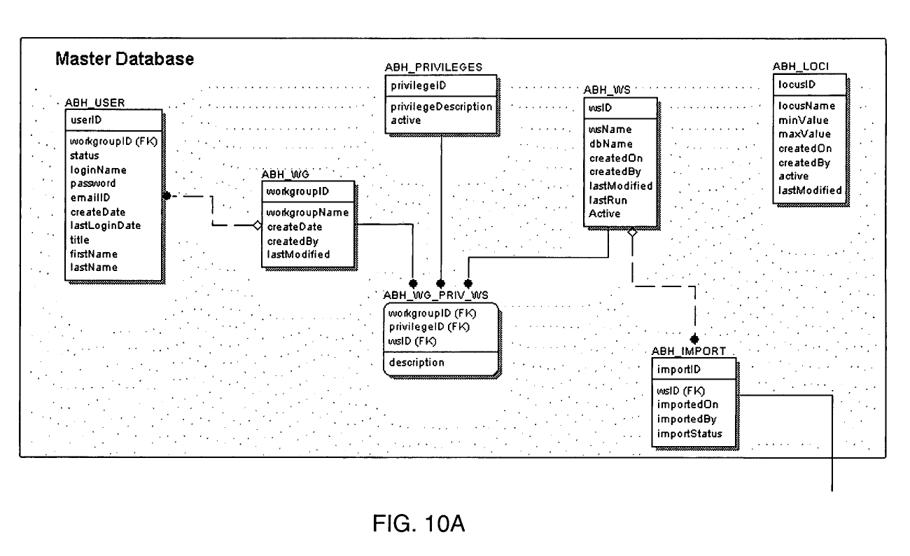

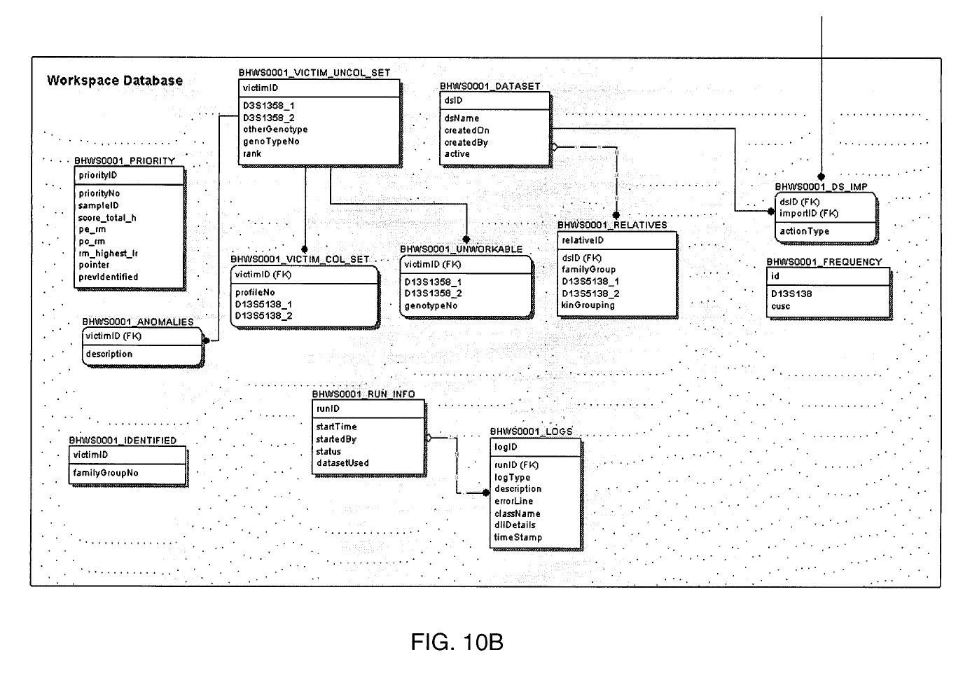

5. Data mapping: User operations and analyses are organized separately in specific tables. FIGS. 10A and B is an illustration of the data mapping procedure in accordance with the invention.

Statistical Analysis:

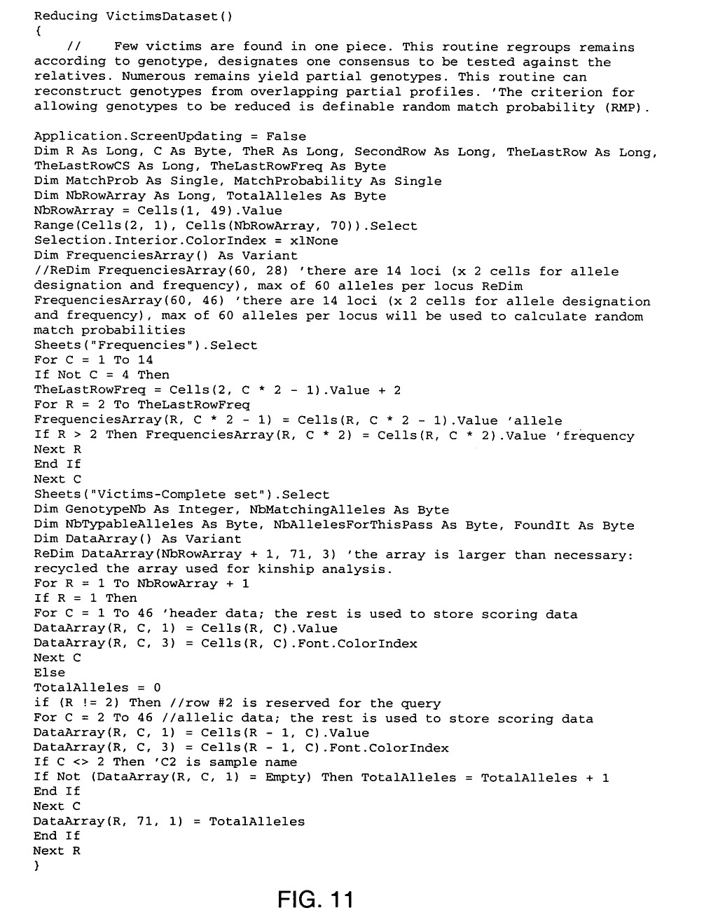

A reduction algorithm which compiles very large collections of complete/partial STR (Short Tandem Repeats), mtDNA (mitochondrial DNA), and SNP (single nucleotide polymorphism) genotypes derived from the remains into a restricted number of consensus genotypes believed to reflect the many different victims. FIG. 11 is an illustration of the reduction algorithm in accordance with the invention.

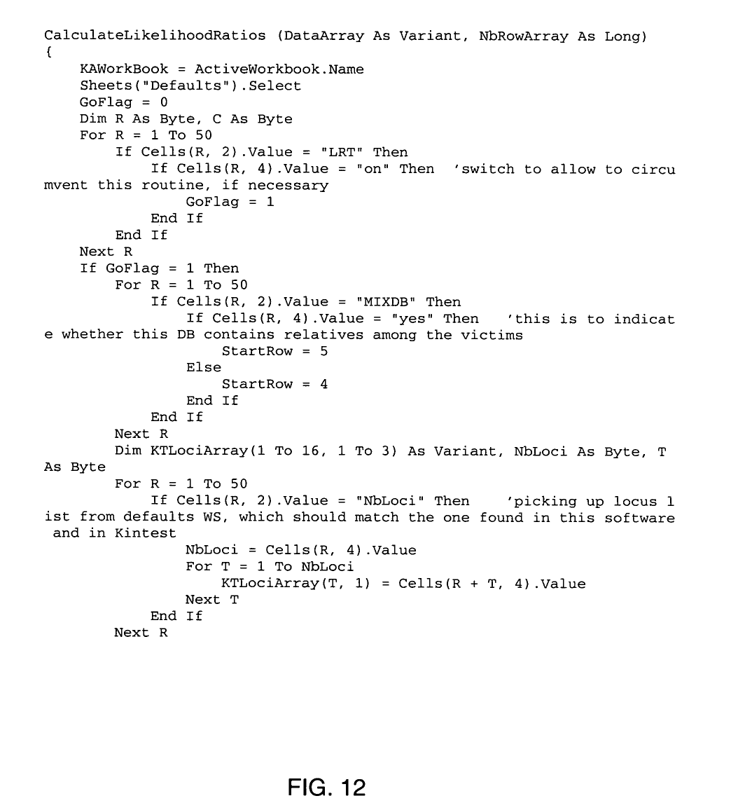

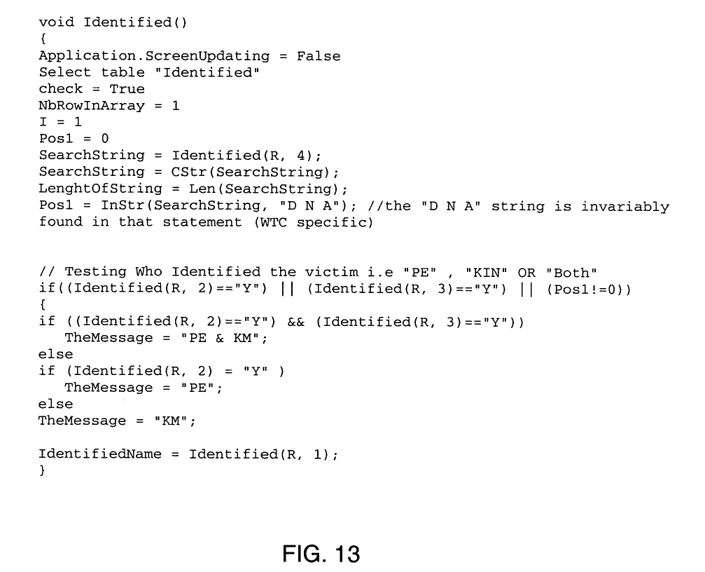

An algorithm which calculates Kinship Indices through a segregated score approach and likelihood ratio calculations. FIG. 12 is a pseudo-code description of the kinship index algorithm in accordance with the invention. An algorithm which compares the consensus genotypes believed to reflect the many different victims against the data set of genotypes from next-of-kin as well as those derived from biological material recovered from personal effects purported to having belonged to the victims for direct matches to personal effects, for evidence of genetic relatedness to kin through the Kinship Indices, or for successful production of a parentage trio with any two members of the next-of-kin cohort to all genotypes derived from samples collected from next-of-kin. No assumptions are made as to the accuracy of the collected information for the submitted reference samples with regards to the reported biological relationship of a next-of-kin or the ownership of a personal effect. As such, associations between genotypes are inferred solely on genetic evidence, the accessory sample identification information being verified for consistency only in later steps of the process. The remains' genotypic data are reduced by the software to generate groups of remains sharing the same partial/complete genotype. The matching process of two genotypes will make either a complete match, as expected between the genotypes derived from a personal effect and remains, or a partial match, as expected between genotypes of a victim and his relatives. A probability calculation is reported for each match. FIG. 13 is a pseudo-code description of comparison algorithm in accordance with the invention.

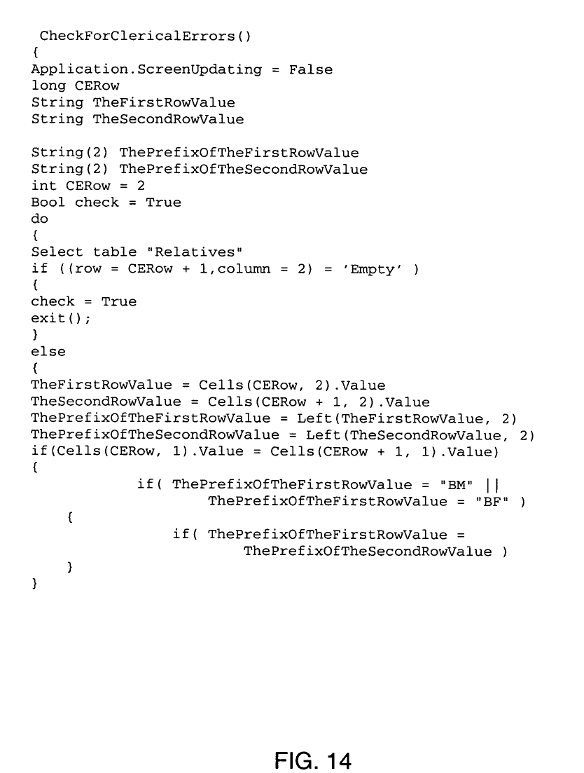

6. Data consistency analyses: An algorithm which cross-checks matches to parentage trios against other kinship scoring results between the victim and other members of the same family to confirm that the purported family structure is consistent with Mendelian inheritance rules. A positive match to a parentage trio is cross-checked by the software against other kinship scoring results between the victim and other members of the same family to confirm that the purported family structure is consistent with Mendelian inheritance rules. FIG. 14 is a pseudo-code description of the data consistency algorithm in accordance with the invention.

The system and method of the present invention synthesizes data and presents them to the end-user, a process that effectively transforms data into information, enhances and optimizes the end-user decision-making processes.

Comprehensive reports: The graphical interface provides a comprehensive and highly organized data report about a dataset, retrieving data from local and/or remote database. Report data are integrated from local and remote data sources. Reports are triggered by the user through the graphical interface when clicking on an object linking to the reports.

Pedigree report: A schematic diagram describing the relationship of the relatives to a matched victim. A schematic diagram is plotted describing the relationship of the victim within the purported family pedigree.

Responsive and interactive graphic components: A user interface that is accessible to a broader range of users. The user interface is the container for this desktop-deployable application that enables use to a broader range of individuals.

The kinship analysis application compares each victim's genotype to all genotypes derived from samples collected from next-of-kin, as well as those derived from biological material recovered from personal effects purported to having belonged to the victims. Associations between genotypes are inferred solely on genetic evidence, the accessory sample identification information being verified for consistency only in later steps of the process.

The remains' genotypic data are reduced by the software to generate groups of remains sharing the same partial/complete genotype.

The computing process, to which each unique genotype identified within the remains data set is subjected, is divided into four steps:

The first step consists of identifying, within the interrogated data set of next-of-kin and personal effects, those genotypes that either show a perfect match (i.e. personal effect, identical twin) to the query (remains), or share at least one allele per tested locus as expected from parent:offspring relationships (herein referred to as the Parent:Offspring Criteria, or POC); likelihood ratio data for each pair wise comparison will be provided for parent-child, full sibling and half sibling putative relationships;

The second step consists of identifying, among the entries that have emerged from step #1 as being significant, those who produce a conclusive parentage trio, that is, all alleles from the purported offspring genotype are accounted for with the two purported parents genotypes;

In the third step, the sample identification information associated with the samples that have produced a conclusive parentage trio is checked for concordance between the reported biological relationships and those suggested by the successful parentage scenario;

Lastly, the reported scores from all other available family members for the victim are checked for scoring consistency within the purported family pedigree. The resulting software tool is a robust, desktop-deployable application that incorporates sensitive routines and can be expanded to other forms of analysis (i.e., mitochondrial DNA analysis).

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A method of kinship analysis for the identification process in mass disasters and missing persons comprising: providing at least one database comprising DNA data, wherein said DNA data includes STR and/or mtDNA and/or SNP data; reducing total remains list into a list of unique victims' genotypes; receiving at least one query; and displaying a first set of genotyping data and a second set of genotyping data, wherein each set of genotyping data is displayed as a graphical table or pedigree diagram and said tables and diagram are linked to statistical likelihood of kinship association.

2. The method according to claim 1, wherein said at least one query comprises genotyping data.

3. The method according to claim 1, wherein said at least one query comprises statistical results linking genotypes.

4. The method according to claim 1, wherein said kinship association comprises identification of all possible parentage trio combinations.

5. The method according to claim 4, wherein parentage trios combinations are determined from Kinship Indices.

6. The method according to claim 4, wherein Kinship indices are obtained from a segregated score approach and likelihood ratio calculations using genetic information of remains and purported family members.

7. The method according to claim 1, wherein said genotyping data is a reduced list of aggregated matching samples.

8. The method according to claim 1, wherein said at least one query comprises a request to display the most likely candidates for a match.

9. The methods according to claim 8, wherein most likely candidates are identified based on match count criteria, allowed discrepancies criteria, likelihood of shared loci criteria, or ignore allelic dropout criteria, or combination thereof.

10. The method according to claim 4, wherein most likely candidates are cross-checked for consistency according to Mendelian inheritance rules.

11. The method according to claim 1, further comprising a plurality of types of genotyping data, wherein each type of said plurality of genotyping data is displayed as a graphical table.

12. The method according to claim 1, wherein said at least one database is a relational database.

13. The method according to claim 1 wherein said pedigree diagram is displayed as a cartoon.

14. The method according to claim 1, wherein said first type of genotyping data is obtained from a first experimental technique and said second type of genotyping data is obtained a second experimental technique.

15. The method according to claim 14 wherein said first experimental technique is Short Tandem Repeats.

16. The method according to claim 14 wherein the genotyping data obtained from said first experimental technique and said second experimental technique is displayed as a graphical table.

17. A computer system comprising: a database including genotyping data, wherein genotyping data includes STR data; and a user interface capable of displaying a first type of genotyping data and a second type of genotyping data, wherein each type of said genotyping data is displayed as a graphical table or pedigree diagram

18. A computer program product comprising a computer-readable medium having computer-readable program code embodied thereon relating to a database including genotyping data, the computer product comprising computer readable program code for effecting the following steps within a computer system: collapsing matching profiles into an aggregate list, receiving at least one query; and displaying a first type of genotyping data and a second type of genotyping data, wherein each type of said genotyping data is displayed as a graphical table or pedigree diagram.

19. The computer program product according to claim 18 further including an algorithm capable of distributing processes across multiple computer systems.

20. A method of displaying genotyping data comprising: a means for providing at least one database comprising genotyping data, wherein said genotyping data includes STR data; a means for receiving at least one query; and a means for displaying a first type of genotyping data and a second type of genotyping data, wherein each type of genotyping data is displayed as a graphical table or pedigree diagram
